(19) United States
(12) Patent Application Publication
(10) Pub. No.: US 2010/0330161 A1
(43) Pub. Date: Dec. 30, 2010

(54) Oral delivery of tetanus toxoid

(75) Inventors: Robert Patrick Doyle, Syracuse, NY (US); Anthony Vortherms, Syracuse, NY (US)

Correspondence Address: HESLIN ROTHENBERG FARLEY & MESITI PC, 5 COLUMBIA CIRCLE, ALBANY, NY, 12203, US

(73) Assignee: SYRACUSE UNIVERSITY TECHNOLOGY TRANSFER AND INDUSTRIAL DEVELOPMENT OFFICE, Syracuse, NY (US)

(21) Appl. No.: 12/803,506

(22) Filed: Jun. 29, 2010

Related U.S. Application Data
(60) Provisional application No. 61/221,226, filed on Jun. 29, 2009.

(51) Int. Cl. A61K 39/385; C07K 14/33; A61P 31/04; A61K 9/127

(52) U.S. Cl. 424/450; 530/409; 424/197.11

(57) ABSTRACT

Orally active vitamin B₁₂-tetanus toxoid (TT) conjugates are described. Attachment of TT to vitamin B₁₂ provides for uptake of the conjugate from the digestive tract, and allows for delivery of the immunologically active TT to the blood. Pharmaceutical compositions and methods of disease prevention employing the orally active Vitamin B₁₂-TT conjugate of the invention are also described.

### CROSS-REFERENCE TO RELATED APPLICATIONS

This application claims priority under 35 U.S.C. §119 to U.S. Provisional Application No. 61/221,226, filed Jun. 29, 2009, the entire disclosure of which is incorporated herein by reference.

### BACKGROUND OF THE INVENTION

The present invention generally relates to Vitamin B₁₂ conjugates for oral delivery of therapeutically useful proteins. More particularly, the present invention relates to an orally active complex of Vitamin B₁₂ conjugated to tetanus toxoid (TT). Methods of preparing and using an orally active Vitamin B₁₂-TT conjugate in prevention of disease are described.

Clostridium tetani is an anaerobic bacterium that is responsible for about 257,000 neonatal deaths a year according to a 2009 report by the US Center for Disease Control. The bacteria enter the body as spores often through flesh wounds including scission of the placental cord. Once in the anaerobic environment of the body, the bacteria produce a toxin, known as tetanospasmin. Tetanospasmin is a 150 kDa protein consisting of a heavy chain (100 kDa) and a light chain (50 kDa) connected via a single disulfide bond. The heavy chain is further divided into a C domain and an N domain. The toxin is one of the most lethal observed to date; with an LD₅₀ of [2.5 ng/kg] in humans. The toxin targets neurons, preventing the release of gamma-aminobutyric acid, which inhibits the neuronal signal for muscle relaxation causing uncontrolled muscle contractions.

The lethality of the tetanus toxin has made vaccination critical worldwide. A significant number of tetanus deaths still occur however, mainly in the developing world, as a result of a lack of vaccination coverage. Developing an oral tetanus vaccine would help solve this problem in such countries. Current attempts at making oral tetanus vaccinations include encapsulation of the tetanus toxoid (TT) and partial expression of TT by bacteria. Unlike other oral vaccines that protect against intestinal infections, an oral tetanus vaccine must target the tetanus toxin, which is only present in the blood. It is therefore important to get the TT across the enterocyte and trigger a significant antibody response. To successfully confer immunity an IgG antibody response must be generated. IgG antibodies are the primary antibodies present in the blood, and therefore are the antibodies needed should a tetanus infection occur. Thus, an oral B₁₂-TT conjugate for oral/enteric delivery that exploits the B₁₂ dietary uptake pathway to deliver TT to blood serum is an attractive means for producing clinically relevant antibody titer.

Specific uptake mechanisms exist in the gastrointestinal tract for uptake of dietary molecules. In the case of Vitamin B₁₂, a specific binding protein is released into the intestine which binds to its ligand in the lumen of the gut. Mammals have a transport mechanism for the absorption and cellular uptake of the relatively large Vitamin B₁₂ molecule which relies upon complexing to a naturally occurring transport protein known as Intrinsic Factor (see FIG. 1B) (Chemistry and Biochemistry of B₁₂, Chapters 16 (Intrinsic Factor, Haptocorrin and their receptors) and 17 (Transcobalamin II), Banerjee, Ruma (Ed), Wiley Interscience 1999; Vitamin B₁₂ Zagalak, et al., (Eds), de Gruyter Press 1979). Russell-Jones et al. (see, e.g., U.S. Pat. Nos. 5,428,023 & 5,807,832) have demonstrated the oral delivery of the proteins erythropoietin (EPO) and granulocyte-colony-stimulating factor (GCSF) through the dietary uptake pathway of vitamin B₁₂ (B₁₂). This work demonstrated that certain proteins up to 34 kDa could be successfully carried through the gastrointestinal tract (GIT) and delivered to blood serum using B₁₂ conjugation. Russell-Jones et al. teach attachment of the luteinizing hormone releasing hormone analog to Vitamin B₁₂ at a carboxyl group of an acid-hydrolyzed propionamide side chain (see FIG. 1A).

Other proteins and peptides have also been conjugated to Vitamin B₁₂ in attempts to provide effective oral delivery compositions. For example, U.S. Pat. No. 5,574,018 teaches Vitamin B₁₂ conjugated to erythropoietin, granulocyte colony stimulating factor and consensus interferon through covalent binding at the primary hydroxyl site of the ribose moiety of the Vitamin B₁₂. Conjugates of other bioactive agents and Vitamin B₁₂ are taught by Grissom et al. (WO 01/30967 & WO 98/08859). Grissom et al. teach covalent attachment of cancer treatment drugs to the cobalt atom of Vitamin B₁₂. In some cases, see e.g., U.S. Pat. No. 6,482,413, the Vitamin B₁₂ is not directly linked to the target peptide or protein, but rather the Vitamin B₁₂ is linked to micro or nanocapsules containing unconjugated, intact peptide or protein. Although this approach is touted by the patentee as providing better protection against proteolysis and Vitamin B₁₂-mediated transport of larger payloads of biologically active peptide or protein, it presents many more technical issues related to polymer encapsulation technology and inefficient transport of the relatively large particles across the intestinal lining.

Therapeutic proteins are able to treat a wide array of illnesses, including cancer, anemia, haemophilia, and diabetes. The major problem with the use of therapeutic proteins is the common need for invasive, subcutaneous, administration. In much of the developing world, such administration, as required for vaccines against diseases such as tetanus, is not widely available. Moreover, proteins are readily degraded in the gastrointestinal tract (GIT) and are typically too large to passively diffuse across the enterocyte. A delivery system that would allow for the reproducible, clinically relevant oral uptake of proteins would change the field of medicinal chemistry. The ability to maintain biological activity throughout synthesis and the uptake system described herein is dependent upon the nature of the protein that is desired to be delivered.

Despite the theoretical advantages of using a conjugate of Vitamin B₁₂ and TT for example to provide an oral delivery form of TT, to date, no one has been successful in developing such an orally active conjugate.

Accordingly, there remains an important and unmet need for an oral vaccine for disease caused by clostridium tetani (tetanus disease), where adequate levels of immunologically active TT are deliverable into the blood from the intestine using the Vitamin B₁₂-Intrinsic Factor uptake mechanism.

### SUMMARY OF THE INVENTION

An orally active delivery conjugate comprising vitamin B₁₂ coupled to tetanus toxoid (TT) is disclosed in accordance with an aspect of the present invention.

In another aspect, the present invention provides a pharmaceutical composition comprising an orally active vitamin B₁₂-TT conjugate.

In another aspect, the present invention provides a pharmaceutical composition comprising an oral delivery form of TT comprising vitamin B₁₂ covalently coupled to TT, wherein the two are coupled between a dicarboxylic acid derivative of the primary (5′) hydroxyl group of the ribose moiety of vitamin B₁₂ and a surface-accessible lysine residue of TT.

In another aspect, the present invention provides a method of preventing tetanus disease, comprising orally administering to a patient in need thereof an immunologically effective amount of orally active B₁₂-TT conjugate.

Kits are also disclosed in accordance with aspects of the invention. The kits may comprise a pharmaceutical composition of the instant invention, and an instruction sheet for oral administration.

These, and other objects, features and advantages of this invention will become apparent from the following detailed description of the various aspects of the invention taken in conjunction with the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts the structure of Vitamin B₁₂ (cobalamin) with sites (indicated by an asterisk) that are modified by or for peptide attachment.

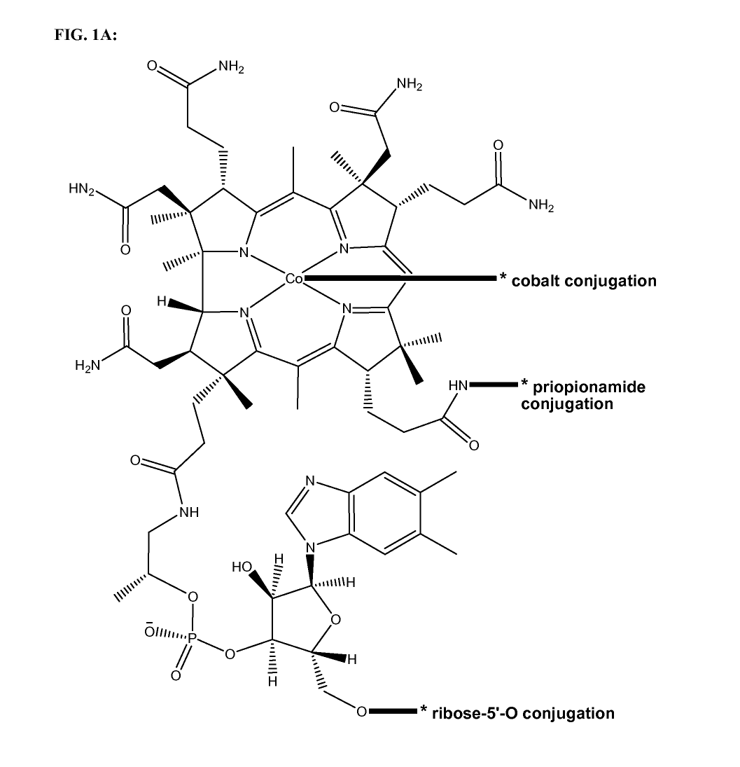

FIG. 1B depicts mammals' active transport mechanism in the gastrointestinal tract (GIT) for the absorption and cellular uptake of the Vitamin B₁₂ molecule.

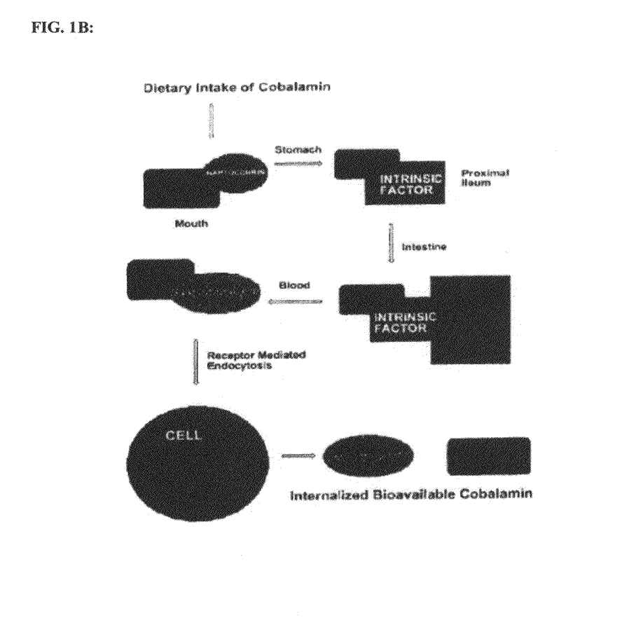

FIG. 2 depicts “Front” and “Top” views of TCII-bound B₁₂ and B₁₂-tethers from MD overlays. Domains in TCII are color-coded: A-domain in red, B-domain in blue, A-B linkage in green. B₁₂ is shown in van der Waals representation. The peptide tether is shown in orange.

FIG. 3 depicts the structure of a conjugated B₁₂ molecule; It was demonstrated that the lysine side chain itself used in the bioconjugation was long enough, when combined with an amide linkage to the 5′ hydroxy position of B₁₂, to not affect B₁₂ binding within TCII in the presence of a tethered chain composed of 10 amino acids (a peptide chain composed of the flexible region of the B-chain of human insulin).

FIG. 4 depicts B₁₂ shown in schematic form, based on newly developed parameter set using AMBER all-atom force field, with important functional motifs identified.

FIG. 5 depicts molecular modeling studies of the tetanus Light Chain (LC) and Heavy Chain (HC).

FIG. 6A depicts the initial time step geometries of the tetanus LC at 300 K and 400K, with important secondary structures labeled.

FIG. 6B depicts the 40 ns time step geometries of the tetanus LC at 300K and 400K, with important secondary structures labeled.

FIG. 7 depicts the solvent-accessible map of tetanus LC lysine side chains (in blue) against surface maps of the entire LC (in yellow, with zinc ions shown in red).

FIG. 8 depicts a representative HPLC of 1, purified on a GF-450-250 tandem column system.

FIG. 9 depicts intrinsic factor-B₁₂ binding assay preformed on B₁₂-TT conjugate.

FIG. 10 depicts the cubilin receptor, which has 27 CUB domains (shown as rectangles) and 8 epidermal growth domains (shown as small ovals) CUB domains 5-8 involved primarily in IF-B₁₂ binding. Large ovals represent Megalin; RAP=receptor associated protein. Cubilin is not a transmembrane protein, so it requires a coreceptor, megalin, which can facilitate endocytosis of cubilin-IF-B₁₂.

FIG. 11 depicts rhenium based fluorescent B₁₂ probe B₁₂-BQBA-[Re(CO)₃]⁺ (2). The probe is attached to vitamin B₁₂ at the 5′-OH of the ribose group.

FIG. 12 depicts cell binding and internalization of 2-IF after approximately 45 minutes of incubation taken at 63× showing images of BeWo a) collected by a monochromatic transmitted light photomultiplier tube (TMPT-1); b) after excitation at 488 nm with fluorescent green emission at ˜560 nm consistent with rhenium(I); c) the merged images after simultaneous scans showed illumination in the nucleus and in the cytosol.

FIG. 13 depicts random depth laser optical slices at ˜1 μm per slice of BeWo cells show fluorescence in the middle slices of the cell confirming internalization of 2-IF after 45 minutes of incubation at 37° C.

FIG. 14 depicts 63× enlarged images of BeWo cells with internalized B₁₂-TT-CypHer 5E (purple) conjugate.

FIG. 15 depicts immunostaining of BeWo cells with fluorescently-tagged anticubilin antibody. The cells show binding to the surface and some internalization of CubAb₄₀₅.

FIG. 16 depicts uptake of 1 tagged with CypHer 5E fluorescent dye (1_{C5E}).

FIG. 17 depicts uptake of 1F₄₀₅-B₁₂ in BeWo cells followed by confocal microscopy.

FIG. 18 depicts colocalization of 1_{C5E} (red) and IF₁₀₅ (blue). Areas of colocalization are shown in purple.

FIG. 19 depicts uptake of 2_{C5E} (red) and IF₄₀₅ (blue) in BeWo cells. Areas of colocalization are shown in purple.

### DETAILED DESCRIPTION OF THE INVENTION

The present invention is based on the observation that TT is a sound target for B₁₂ conjugation and subsequent delivery. The protein can withstand high temperatures and still retain its conformation and antigencitiy/immunogenicity. This is demonstrated by the ability of anti-TT antibodies (polyclonal and monoclonal) to bind the conjugate. Furthermore, conjugation of the toxoid to vitamin B₁₂ does not eliminate antigenicity of the toxoid.

In one aspect, Applicants' invention relates to an orally active vitamin B₁₂-tetanus toxoid (TT) conjugate.

In one aspect, the TT may be covalently linked to Vitamin B₁₂, either directly or through one or more linkers.

In a particular aspect, for the orally active vitamin B₁₂-TT conjugate, the TT is covalently attached to the primary (5′) hydroxyl group of the ribose moiety of vitamin B₁₂.

In some embodiments, for the orally active vitamin B₁₂-TT conjugate, vitamin B₁₂ is directly or indirectly attached to TT via surface-accessible lysine residues on TT only.

In some embodiments, for the orally active vitamin B₁₂-TT conjugate, any attached Vitamin B₁₂ molecule resides on the exterior of one or more of the tetanus subunits such that the overall tetanus geometry is maintained.

In some embodiments, the orally active vitamin B₁₂-TT conjugate exhibits at least a portion of the immunogenicity of TT. In some embodiments, this immunogenicity remains even after the conjugate enters the patient's bloodstream.

In some aspects of the invention, the vitamin B₁₂ and TT of the conjugate are attached via a carbamate-linkage.

In some aspects, in the conjugate there is one or more spacer groups between the vitamin B₁₂ and TT.

In certain embodiments, there may be a spacer group between the vitamin B₁₂ and TT, and this spacer group may comprise a polyethylene glycol monomer spacer unit.

In some embodiments, there may be a spacer group between the vitamin B₁₂ and TT, and this spacer group may comprise two or more polyethylene glycol monomer spacer units.

In another aspect, the present invention relates to a pharmaceutical composition containing an orally active vitamin B₁₂-TT conjugate.

In certain embodiments, the pharmaceutical composition of the present invention comprises both an orally active vitamin B₁₂-TT conjugate and a pharmaceutically acceptable carrier and/or diluent.

In some aspects, the pharmaceutical composition of the present invention comprises both an orally active vitamin B₁₂-TT conjugate, and Intrinsic Factor. In some embodiments, the Intrinsic Factor is human Intrinsic Factor.

In some embodiments, the present invention relates to an oral delivery form of TT comprising vitamin B₁₂ covalently coupled to TT, wherein the covalent coupling is between a dicarboxylic acid derivative of the primary (5′) hydroxyl group of the ribose moiety of vitamin B₁₂ and surface-accessible lysine residues of TT, and a pharmaceutically acceptable carrier suitable for oral delivery. Preferably, in such embodiments the pharmaceutical composition provides an immunologically effective does of TT when delivered orally to a mammal.

The present invention also relates to a method of preventing tetanus disease, comprising orally administering to a patient an immunologically effective amount of an orally active B₁₂-TT conjugate. In such embodiments, the present invention may represent an orally-administered tetanus vaccination.

In some embodiments, the present invention is administered as an orally active tetanus vaccine, which comprises a B₁₂-TT conjugate, and the TT is covalently attached to the primary (5′) hydroxyl group of the ribose moiety of vitamin B₁₂.

The immunogenic compositions of the present invention, such as those including a vitamin B₁₂-TT conjugate, may be administered in combination with an antibiotic treatment regime. In one embodiment, the antibiotic is administered prior to administration of the vitamin B₁₂-TT conjugate of the invention or the composition comprising the vitamin B₁₂-TT conjugate of the invention.

The present invention also relates to kits. Kits may comprise an orally active conjugate of the instant invention, or pharmaceutical compositions of the present invention, and instruction sheet(s) for oral administration.

In practicing the present invention, many conventional techniques in chemistry and molecular biology are used. Such techniques are well known and are explained in more detail in, for example, Hermanson, G. T. Bioconjugate Techniques Academic Press: San Diego, Calif., 1996 and Sambrook, J. and Russel, D. W. Molecular Cloning: A Laboratory Manual, 3rd ed.; Cold Spring Harbor Laboratory Press: Woodbury, N.Y., 2003; Vol. 3. The contents of these and other publications referenced herein are hereby incorporated by reference into the present disclosure.

### Definitions

“Bioactive molecules” or “biologically active substances” include proteins, peptides, hormones, small molecule drugs, haptens, antigens, antibodies.

“Orally active” means that a compound or molecule is therapeutically active when administered orally. The term is also intended to encompass those compounds (for example, tetanus toxoid), the therapeutic action or activity of which is to stimulate a protective immune response. The vitamin B₁₂-polypeptide conjugates of the present invention are orally active because, when orally administered, the conjugates retain their immunogenic character and are able to elicit an antibody response. Thus, when describing immunogenic compositions of the present invention, such as the vitamin B₁₂-TT conjugates, persons having ordinary skill in the art will understand that, when used in preventing or protective against disease, an immunologically effective amount of said compositions would be used. An “immunologically effective amount” is an amount which, when administered to an individual, either in a single dose or as part of a series, increases a measurable immune response or prevents or reduces a clinical symptom.

“Vitamin B₁₂ conjugate” refers to the bioactive molecule or biologically active substance covalently linked to Vitamin B₁₂, either directly or through one or more linkers.

The term “method of preventing”, when used in connection with the present invention, includes active immunization for the prevention or protection against the identified disease. “Prevention” is intended to mean, for example, to prevent or inhibit the replication of the bacteria which cause the identified disease, to inhibit transmission of the bacteria, or to prevent the bacteria from establishing itself in a host, or to alleviate the symptoms of the disease caused by infection. “Protecting”, as used herein with respect to the present invention, means that the conjugate prevents or reduces the onset of, or symptoms of, the disease caused by the organism from which the antigen(s) (e.g., TT) used in the treatment (e.g., vaccine) is derived.

### Administration

In a further embodiment of the invention there is provided a medicament which comprises a conjugate according to the invention together with a pharmaceutically acceptable carrier or diluent.

Examples of pharmaceutically acceptable carriers and diluents include typical carriers and diluents such as sodium bicarbonate solutions and similar diluents which neutralize stomach acid or have similar buffering capacity, glycols, oils, oil-in-water or water-in-oil emulsions, and include medicaments in the form of emulsions, gels, pastes and viscous colloidal dispersions. The medicament may be presented in capsule, tablet, slow release or elixir form or as a gel or paste. Furthermore, the medicament may be provided as a live stock feed or as food suitable for human consumption.

Pharmaceutically acceptable carriers include conventional excipients such as binders, including gelatin, pre-gelatinized starch, and the like; lubricants, such as hydrogenated vegetable oil, stearic acid and the like; diluents, such as lactose, mannose, and sucrose; disintegants, such as carboxymethyl cellulose and sodium starch glycolate; suspending agents, such as povidone, polyvinyl alcohol, and the like; absorbents, such as silicon dioxide; preservative, such as methylparaben, propylparaben, and sodium benzoate; surfactants, such as sodium lauryl sulfate, polysorbate 80, and the like; and colorants, such as F.D & C. dyes and the like.

Pharmaceutically acceptable carriers may be either solid or liquid form. Solid form preparations include powders, tablets, dispersible granules, capsules, and cachets. A solid carrier is suitably one or more substances which may also act as diluents, flavoring agents, solubilizers, lubricants, suspending agents, binders or tablet disintegrating agents. The solid carrier material also includes encapsulating material. In powders, the carrier is finely divided active compounds. In the tablet, the active compound is mixed with the carrier having the necessary binding properties in suitable proportions and compacted in the shape and size desired. Suitable solid carriers include, but are not limited, to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Delivery may use a sustained release form.

Liquid form preparations include solutions, suspensions, and emulsions. Aqueous solutions suitable for oral use are prepared by dissolving the active component in water or other suitable liquid and adding suitable colorants, flavors, stabilizing agents, and thickening agents as desired. Aqueous solutions suitable for oral use may also be made by dispersing the finely divided active component in water or other suitable liquid with viscous material, such as natural or synthetic gums, resins, methylcellulose, sodium carboxymethylcellulose, and other suspending agents known in the art.

Also included are solid form preparations which are intended to be converted, shortly before use, to liquid form preparations for either oral or parental administration. Such liquid forms include solutions, suspensions, and emulsions. These particular solid form preparations are provided in unit dose form and as such are used to provide a single liquid dosage unit. Alternatively, sufficient solid preparation may be provided so that the after conversion to liquid form, multiple individual liquid doses may be obtained by measuring predetermined volumes of the liquid form preparation as with a syringe, teaspoon, or other volumetric measuring device.

Other components of the pharmaceutical compositions of the invention can include pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

The solid and liquid forms may contain, in addition to the active material, flavorants, colorants, stabilizers, buffers, artificial and natural sweeteners, dispersants, thickeners, solubilizing agents, and the like. The liquid utilized for preparing the liquid form preparation is suitably water, isotonic water, ethanol, glycerin, propylene glycol, and the like, as well as combinations thereof. The liquid utilized will be chosen with regard to the route of administration.

In some embodiments, the conjugate may be administered as a chewing gum. The conjugate may be included in a known chewing gum composition such as those described in U.S. Pat. No. 7,078,052, which is incorporated herein by reference. The chewing gum can be low or high moisture, sugar or sugarless, wax containing or wax free, low calorie (via high base or low calorie bulking agents), and/or may contain dental agents.

Chewing gum generally consists of a water insoluble gum base, a water soluble portion, and flavor. The water soluble portion contains the conjugate and optionally flavor and dissipates with a portion of the conjugate over a period of time during chewing. The gum base portion is retained in the mouth throughout the chew.

The insoluble gum base generally comprises elastomers, resins, fats and oils, softeners and inorganic fillers. The gum base may or may not include wax. The insoluble gum base can constitute approximately 5% to about 95% by weight of the chewing gum, more commonly the gum base comprises 10% to about 50% of the gum, and in some preferred embodiments approximately 25% to about 35%, by weight, of the chewing gum.

Preferably, the preparations are unit dosage form. In such form, the preparation is subdivided into unit doses containing appropriate quantities of the active components. The unit dosage form can be a packaged preparation, such as packaged tablets or capsules. The unit dosage can be a capsule, cachet, or tablet itself or it can be the appropriate number of any of these in packaged form.

The quantity of active material in a unit dose of preparation is varied according to the particular application and potency of the active ingredients.

Embodiments of the invention provide a method of delivering an active substance to any uni- or multicellular organism, including bacteria, protozoa, or parasites, which has a requirement for Vitamin B₁₂ as well as a specific uptake mechanism for the same, which method comprises administering a conjugate of the invention to the organism.

One way of assessing efficacy of prophylactic treatment involves monitoring immune responses against the TT in the compositions of the invention after administration of the composition.

Another way of assessing the immunogenicity of the component proteins of the immunogenic compositions of the present invention is to express TT proteins recombinantly and to screen patient sera or mucosal secretions by immunoblot. A positive reaction between the protein and the patient serum indicates that the patient has previously mounted an immune response to the protein in questions, i. e., the protein is an immunogen.

Another way of checking efficacy of oral delivery involves monitoring C. tetani infection after administration of the compositions of the invention. One way of checking efficacy of prophylactic treatment involves monitoring immune responses both systemically (such as monitoring the level of IgG1 and IgG2a production) and mucosally (such as monitoring the level of IgA production) against TT after administration of compositions of the present invention.

The vaccine compositions of the present invention can be evaluated in in vitro and in vivo animal models. Particularly useful mouse models include those in which oral immunization is followed by challenge with C. tetani or intranasal challenge.

The efficacy of immunogenic compositions of the invention can also be determined in vivo by immunizing animal models (e.g., guinea pigs or mice) with the immunogenic compositions and ascertaining the level of protection obtained after challenge with tetanus toxin.

### Molecular Modeling Studies of Tetanus, Cyanocobalamin Force Field Parameterization Development, And B₁₂-Bioconjugate Design Studies

The computational efforts used to address design and interaction considerations in Applicants' experimental tetanus studies have progressed significantly since Applicants' original B₁₂-Insulin study, which demonstrated that a proper force field parameterization for B₁₂ produced an accurate binding interaction between B₁₂ and its transport protein transcobalamin II (TCII), an important result that enables the more general study of all B₁₂ bioconjugates. The most recent published work from the computational studies incorporated a final B₁₂ force field parameterization with long molecular dynamics (MD) simulations to provide atomic-level descriptions of both B₁₂-TCII binding and best-practices approaches for bioconjugate design based on the B₁₂ transport. Along with new insights into the mode of B₁₂ binding in TCII and potential approaches for subsequent bioconjugate design, this study revealed minimum criteria for productive bioconjugate linkages between B₁₂ and most any other molecule (pharmaceutical or otherwise) and describes in detail how steric interactions must be minimized between the linked molecule and (in this case) TCII to retain B₁₂ binding for delivery. These first two studies effectively solidified all of the force field development for B₁₂ and its bioconjugates and demonstrated the utility of molecular modeling in all subsequent bioconjugate design work. Applicants' most recent MD simulation work on the tetanus light chain has revealed a great deal about both the stability of this small fragment (important for demonstrating that the computational work reproduces the stability observed in tetanus during vaccine manufacturing) and the possible distribution of B₁₂ around the light chain based on the positions of solvent-accessible lysines.

Completion of Force Field Parameter Development: The initial MD study of the B₁₂-Insulin bioconjugate (Petrus A. K., Allis D. G., Smith R. P., Fairchild T. J., and Doyle R. P. “Exploring The Implications Of Vitamin B12 Conjugation To Insulin On Insulin Receptor Binding And Cellular Update.” ChemMedChem, 4(3) (2009) 421-426) demonstrated that the force field parameters for B₁₂ using the GROMOS96 force field were adequate for modeling its interaction with TCII, a key step in subsequent molecular modeling studies. Over the course of this initial study and during the time between submission and publication, two additional B₁₂-specific papers were published that reported additional parameterization development for the AMBER, CHARMM, and GROMOS96 molecular mechanics force fields. The great consistency in the electrostatic calculations, combined with the higher level of theory used in these newer studies, warranted their use as the production-grade charge values (other force field parameters were retained from the original study) for a more generic B₁₂ bioconjugate study (Allis D. G., Fairchild T. J., and Doyle R. “The Binding of Vitamin B12 to Transcobalamin(II); Structural Considerations for Bioconjugate Design—a Molecular Dynamics Study.” Molecular Biosystems, accepted) that specifically addressed design considerations for B₁₂ bioconjugates and, over the course of very long-duration MD simulations, showed the stability of B₁₂ in TCII and the importance of B₁₂ in stabilizing the observed crystal geometries of both TCII and Intrinsic Factor (IF).

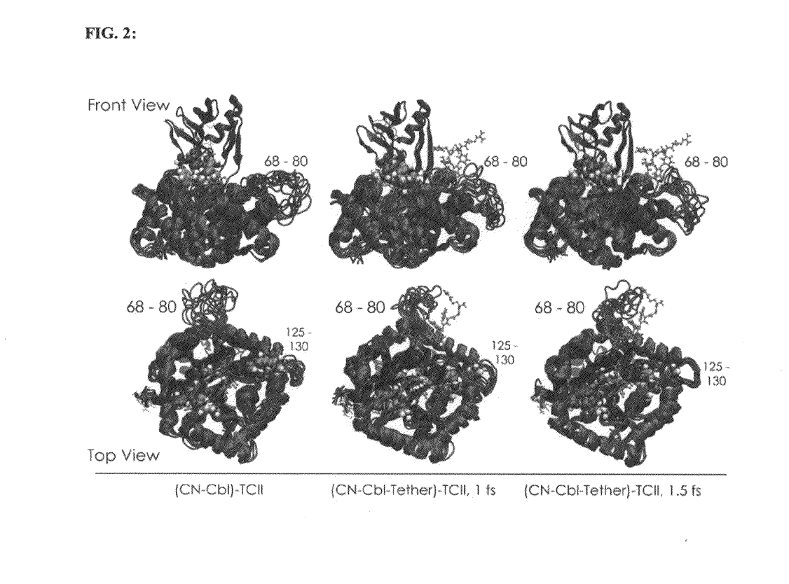

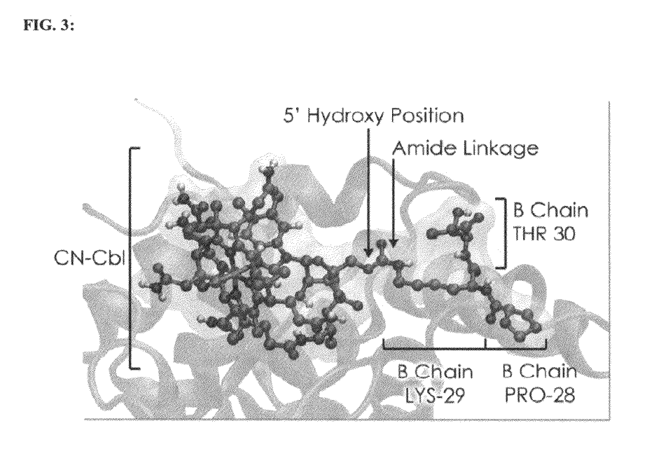

The generation of time-averaged snapshots of the TCII/B₁₂-tether complex (see FIG. 2) provide information about the interactions between B₁₂-tethered molecules and the TCII transport protein. Based on such analyses, the steric demands of TCII (as well as IF and, very likely, happtocorrin) can be understood in order to determine what length of linkage between B₁₂ and its conjugate is required to not affect B₁₂ binding (the retention of B₁₂ in its binding pocket being key to the entire delivery mechanism). By generating a time-averaged geometry from all of the MD simulations, it was determined that the lysine side chain itself used in the bioconjugation was long enough, when combined with an amide linkage to the 5′ hydroxy position of B₁₂ (see FIG. 3), to not affect B₁₂ binding within TCII in the presence of a tethered chain composed of 10 amino acids.

The results of this MD study have proven to be of considerable importance both for any subsequent B₁₂ bioconjugate experiments employing tethered molecular designs and for identifying the critical importance of accounting for steric interactions between transport protein and bioconjugate. In the case of tetanus, both a considerably large and very stable macromolecule, this computational work helps to either identify candidate positions where B₁₂ binding to its transport proteins may not be affected by steric congestion, and also provides atomic-level descriptions of what additional synthetic studies may improve the likelihood of B₁₂ binding by minimizing the strength of interactions between transport proteins and tetanus, as well as the synthetic efforts required to develop new linking groups to facilitate successful B₁₂ binding.

### Vitamin B₁₂ Conjugates

Embodiments of the present invention are directed to complexes which include a bioactive substance linked to at least one carrier molecule which is Vitamin B₁₂ or an adenosylcobalamin, methylcobalamin, cyanocobalamin, aquocobalamin, glutathionylcobalamin, hydroxycobalamin, cyanocobalamin carbanalide, and 5-o-methylbenzylcobalmin ((5-OMeBza)CN-Cbl), as well as the desdimethyl, monoethylamide and the methylamide analogs of all of the above. Also included are the various analogs and homologs of cobamamide such as coenzyme Vitamin B₁₂ and 5′-deoxyadenosylcobalamin. Other analogs include chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin ((Ade)CN-Cbl), cobalamin lactone, cobalamin lactam and the anilide, ethylamide, monocarboxylic and dicarboxylic acid derivatives of Vitamin B₁₂ or its analogs. Both the ability of the Vitamin B₁₂ portion of the conjugate to undergo binding reactions for uptake and transport in a vertebrate host and the activity of the biologically active substance are substantially maintained

Preferred embodiments of the invention are directed to biologically active substances, such as proteins and peptides, covalently linked to Vitamin B₁₂. The biologically active substance-Vitamin B₁₂ conjugate has the advantage that biologically active substance may be administered orally rather than by intravenous injection. It avoids the side effects of other non-invasive routes of administration, such as nasal or pulmonary administration. Administration of the biologically active substance—Vitamin B₁₂ conjugate has the further advantage that a necessary vitamin is co-administered. The biologically active substance—Vitamin B₁₂ conjugate has a long-lived mode of action which is a further advantage, regardless of how the conjugate is administered.

Preferred derivatives of Vitamin B₁₂ include the mono-, di- and tricarboxylic acid derivatives or the proprionamide derivatives of Vitamin B₁₂. Carriers may also include analogs of Vitamin B₁₂ in which the cobalt is replaced by zinc or nickel. The corrin ring of Vitamin B₁₂ or its analogs may also be substituted with any substituent which does not affect its binding to Intrinsic Factor.

In a preferred embodiment of the invention there is provided a covalently linked conjugate comprising a Vitamin B₁₂ covalently linked to TT.

Mammals have an active transport mechanism in the gastrointestinal tract (GIT) for the absorption and cellular uptake of the relatively large Vitamin B₁₂ molecule (1350 Da) Embodiments of the delivery system take advantage of the natural Intrinsic Factor mediated uptake mechanism for dietary B₁₂ to overcome the two major hurdles of enteric delivery, namely protection of biologically active substance from GIT proteolysis and uptake and transcytoses of the enterocyte.

Vitamin B₁₂ binds to Intrinsic Factor (IF) and proceeds down the small intestine where the complex binds to the IF-receptor on the ileum wall. The Intrinsic Factor-Vitamin B₁₂ receptor complex then undergoes endocytosis, releasing Vitamin B₁₂ into the blood serum where it becomes bound to transcobalamin (II) (TCII). Embodiments of the invention adapt this uptake pathway for the delivery of a biologically active substance, such as a biologically active protein (e.g., TT). The recognition of, and affinity for, the various binding Vitamin B₁₂ proteins is maintained. Conjugation to Vitamin B₁₂ protects bound proteins from digestion and also facilitates their internalization and transport into blood serum overcoming the two major hurdles for oral delivery of biologically active substances. In people with impaired Vitamin B₁₂ uptake, research has shown that co-administration of Intrinsic Factor alongside Vitamin B₁₂ greatly increases uptake (WO 03/026674).

In some embodiments, Intrinsic Factor is co-administered along with the Vitamin B₁₂ conjugate to increase uptake of the Vitamin B₁₂-biologically active substance conjugate.

Embodiments of the invention include Vitamin B₁₂ conjugates that can be used to deliver a biologically active substance to any uni- or multicellular organism with a requirement for and a specific transport mechanism for Vitamin B₁₂.

Vitamin B₁₂ also undergoes what is termed “enterohepatic recirculation” from bile salts (Chemistry and Biochemistry of B₁₂, Chapter 15, pages 406-407, Banerjee, Ruma (Ed), Wiley Interscience 1999). This “recycling” of Vitamin B₁₂ is vital in ensuring Vitamin B₁₂ deficiency does not occur. By coupling biologically active substances to Vitamin B₁₂, this recirculation may result in a longer mean residency time for biologically active substance, essentially producing a long-acting biologically active substance. The “side product” of this process will be a dose of Vitamin B₁₂.

Both the attachment point on the protein and the attachment point on the Vitamin B₁₂ must be carefully considered. Attachment to the biologically active molecule must be made without loss of activity or compromising stability. Attachment of the Vitamin B₁₂ to the protein potentially affects the three dimensional structure which in turn may affect biologically active substance effectiveness and stability.

In some embodiments, Vitamin B₁₂ and a biologically active substance are coupled together in such a way that neither molecule is inhibited by the other. Preferably, the vitamin is recognized by the series of enzymes involved in its uptake through the GIT so that the biologically active substance interacts with its receptor to induce the cascade effect. In some embodiments, specific sites on both molecules are chosen for conjugation, wherein the sites are known not to be important for recognition and activity. In some embodiments, the Vitamin B₁₂ and biologically active substance will be coupled directly together. In other embodiments, Vitamin B₁₂ and the bioactive molecule are coupled with a “linker” or “spacer” unit between the two molecules to produce distance between the Vitamin B₁₂ and biologically active substance.

In some embodiments, the spacer units are provided by polyethylene glycol (PEG) monomers. In some embodiments, the mean residence time of the biologically active substance is increased by the use of long chain polyethylene glycol (PEG) units (750-10000 Da). Conjugates of the type Vitamin B₁₂-PEG₇₅₀₋₁₀₀₀₀-biologically active substance are produced.

It has been extensively reported in the literature that PEG conjugates exhibit increased plasma half-lives, improved resistance to proteolysis, reduced immunogenicity and antigenicity compared to parental compounds including proteins (Van Spriel, A. B. et al. 2000 Cytokine 12:666-670; Park, Y. et al. 2002 Bioconjugate Chemistry 13:232-239; Werle, M. et al. 2006 Amino Acids 30:351-367; Pasut, Gianfranco et al. Adv. Polymer Sci. 192:95-134).

In some embodiments, the conjugates are coupled through bifunctional PEG units with a stable bond at the biologically active substance-PEG junction but a reversible bond at the Vitamin B₁₂-PEG junction (e.g., a disulfide bond sensitive to reducing agents in blood serum) to achieve targeted release. This approach provides a longer-lived biologically active substance (compared to non-pegylated forms), which is transported orally. Besides providing for optimization of the spacing between the Vitamin B₁₂ and the biologically active molecule, the PEG linkers provide better uptake and longer lifetime for the biologically active substance.

In some embodiments, the conjugates are coupled through bifunctional PEG units with a stable bond at the biologically active substance-PEG junction but a reversible bond at the Vitamin B₁₂-PEG junction (e.g., a disulfide bond sensitive to reducing agents in blood serum) to achieve targeted release. This approach provides a longer-lived biologically active substance (compared to non-pegylated forms), which is transported orally. Besides providing for optimization of the spacing between the Vitamin B₁₂ and the biologically active molecule, the PEG linkers provide better uptake and longer lifetime for the biologically active substance.

In some embodiments, there is no linker and attachment is directly between Vitamin B₁₂ and biologically active substance. In other embodiments, a linker is used. The linker may be of various lengths. In linker embodiments, the linker may be about 3-150 atoms in length, or about 3-100 atoms in length, or about 3-40 atoms in length. In general, longer linkers improve stability and function as they allow for some distance of the peptide or protein from Vitamin B₁₂ and proper folding of the protein portion of the conjugate. A non-limiting list of suitable coupling agents also include 1,3-diisopropyl-carbodiimide (DIPC), any suitable dialkyl carbodiimide, 2-halo-1-alkyl-pyridinium halides (Mukaiyama reagents), 1-(3-dimethylaminopropyl)-3-ethyl carbodiimide (EDC), propane phosphonic acid cyclic anhydride (PPACA) and phenyl dichlorophosphates, etc. which are available, for example from commercial sources such as Sigma-Aldrich Chemical, or synthesized using known techniques.

As discussed above, in some embodiments one or more PEG monomers are added to optimize the distance between the Vitamin B₁₂ and the biologically active substance.

In some embodiments, the linker may be degradable. The degradation may occur naturally in the body or require the administration of a second factor to trigger degradation of the linker and release of free biologically active substance from the Vitamin B₁₂-biologically active substance complex. Examples of such degradable linkers include disulfide bonds, thioesters, esters, carbamates, and thioethers.

Some major advantages of the instant delivery system of TT include (1) oral delivery; (2) TT's capability of maintaining biological potency when conjugated to B₁₂; (3) successfully absorption of the conjugate into the blood; and (4) the quantity of TT that is required for clinical efficacy is well within the B₁₂ uptake capacity.

In some embodiments, conjugation of Vitamin B₁₂ to the biologically active material is at the 5′-hydroxy group of the ribose unit of the α unit.

In some embodiments, the Vitamin B₁₂-conjugated biologically active substance may be encapsulated in protective liposomes for greater improvement in stability.

Embodiments of the invention may provide for extended release by enterohepatic recirculation of Vitamin B₁₂.

Quantum Chemical Parameterization of Vitamin B₁₂ For The AMBER Force Field. The force field parameterization of B₁₂ is essential to all subsequent molecular dynamics studies of B₁₂-bioconjugates and their complexes with proteins involved in B₁₂ uptake. The results of any force field parameterization process are a set of (most often) harmonic force constants (describing the stiffness of covalent bonds, angles, and dihedral angles/improper torsions) and partial charges (which govern all electrostatic interactions modeled in binding interactions). For virtually all biomolecular force fields (GROMOS, CHARMM, AMBER), these values have been determined in great detail for amino and nucleic acids and not for other molecules of biological relevance. A complete parameter set for a molecule must adequately describe the covalent framework in a manner consistent with the established force constants for other members of the force field (the resulting structures must have responses to the temperature bath of the simulation that are similar to one another) and must have partial charges that are calculated by the same algorithms, as the over/under-polarization of atomic charges along bonds or between molecular fragments can lead to binding interaction strengths between molecules being over/under-estimated.

The B₁₂ molecule has been previously parameterized within the GROMOS formalism as part of initial B₁₂-insulin bioconjugate studies. While this parameterization process was thorough, the GROMOS96 force field relies on a unified-atom approximation that (a) removes all aliphatic (C—H) hydrogen atoms for reasons of computational expediency and (b) slightly under-polarizes the partial charges along covalent bonds in highly polar fragments (O—H, N—H), thereby reducing the strength of binding interactions between molecular complexes. Both points (a) and (b) are of great importance in governing the reliability of long-duration MD simulations. In the case of the B₁₂ binding to its transport protein TCII, long-duration MD simulations revealed that many of the binding pocket interactions identified by crystallographic studies were not as persistent as the diffraction experiments provided evidence for. While the confinement of a protein to a solid-state geometry, by removing the dynamical behavior of proteins and their ligands present in solution, can alter binding geometries, the known limitations of the GROMOS96 force field listed above would lead to these interactions not being predicted to be as strong as the diffraction experiment indicates, requiring that the entire B₁₂ molecule then be reparameterized for and the influence of charge polarization at functional groups responsible for TCII-B₁₂ binding be recalculated.

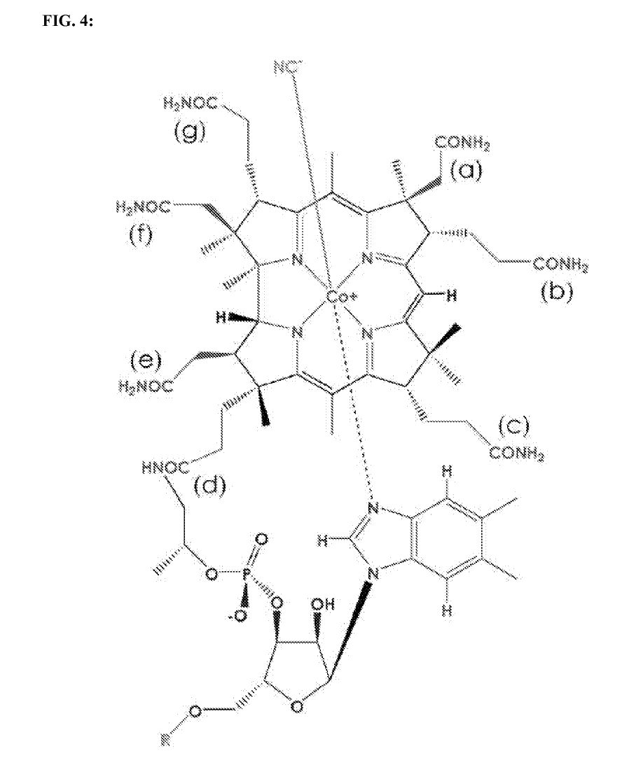

A new parameter set has been developed for B₁₂ with the AMBER all-atom force field (see FIG. 4). The AMBER force field is not only one of the most popular formalisms for biomolecular simulations, but it has also outperformed the GROMOS96 force field in initial in-house studies of one very important class of biomolecule. The enhanced charge polarization employed in the AMBER force field properly reproduces the base pairing interactions of B-DNA over 20 ns MD simulations, whereas the GROMOS96 force field (with its under-polarized hydrogen-bonding interactions) will lead to partial un-pairing of a 12-mer B-DNA duplex over the same time span. While the stronger interactions between functional groups is expected to be of great benefit in completing the proper MD simulations of B₁₂ to all of its transport proteins, this success in modeling comes at the cost of greater simulation times because the force field now includes ALL hydrogen atoms within a protein and ligand. This not only increases the number of atoms in the system but also requires that smaller time-steps be used in the MD simulations because of the rapid (high-frequency) vibrational (stretching) motions of aliphatic C—H hydrogen atoms.

The B₁₂ AMBER parameterization process involves (1) the quantum chemical optimization of B₁₂, (2) the generation of harmonic force constants for bond and angle terms by atomic displacements, and (3) the generation of partial charges using either small-displacement molecular dynamics simulations or full molecular electrostatic potential (MEP) calculations. The MEP calculations for B₁₂ have been completed, providing the first complete AMBER-based charge map of this large biomolecule. Relevant to the binding studies described above, the O—H and N—H bond dipoles in the AMBER calculations increase by approximately 10% over those for the GROMOS96 values, a difference expected to significantly contribute to the retention of diffraction-identified B₁₂ interactions in AMBER-based MD simulations. The generation of new force constants for stretch, bend, and dihedral terms is greatly simplified in B₁₂ because many of the structural motifs found in the covalent framework exist within already-determined nucleic acid and amino acid side chain motifs. The generation of new force field terms (which only consider 2-atom (stretch), 3-atom (bend) and 4-atom (dihedral) interactions, is localized to the cobalt and corrin ring. These final terms are currently being calculated along with the Tetanus MD simulations.

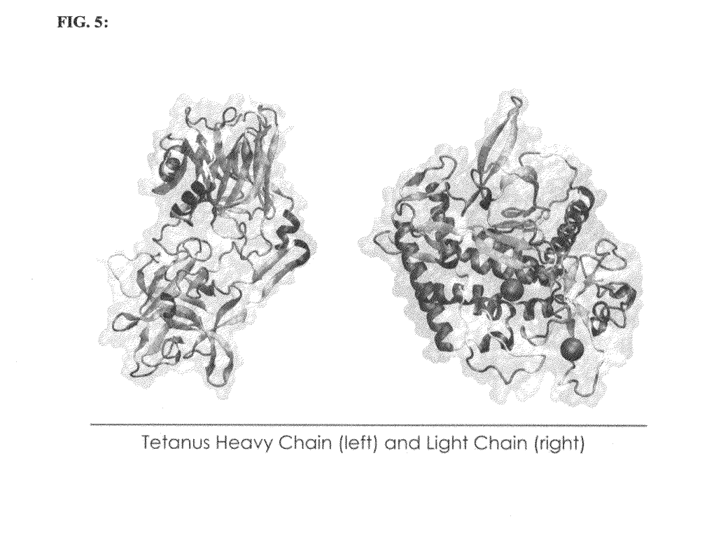

Tetanus Light Chain Thermal Stability Studies: Molecular modeling studies of the tetanus Light Chain (LC) and Heavy Chain (HC) (see FIG. 5) were proposed for this study for two important reasons. First, it is known that the manufacture of tetanus vaccine, despite the use of caustic conditions to most biological proteins (353 K in formalin), is found to not significantly affect the immune response to the vaccine itself, indicating that the protein must either be stable enough to retain its geometry or must refold into the same basic geometry as the native protein. Second, the chemistry involved in the formation of B₁₂ bioconjugates is specific to lysine side chains. In order for a B₁₂ bioconjugate oral tetanus vaccine to work, the overall tetanus geometry must be maintained, meaning that any attached B₁₂ molecule must likely reside on the exterior of one or more of the tetanus subunits.

MD simulations can be used to address both of the above-identified important areas. The first aspect of tetanus stability can be considered using temperature-dependent MD simulations to provide a prediction as to whether or not the tetanus subunits are stable to extreme conditions or if these subunits likely unfold upon heating and then refold upon cooling. Simulations were performed on the tetanus LC, the region specifically responsible for the deleterious effects of tetanus.

Experimental. 1,1′-Carbonyl-di-(1,2,4-triazole) (CDT), Cyanocobalamin (vitamin B₁₂), 2,2′-azido-bis(3-ethylbenzothiazoline-6-sulfonic acid), sodium chloride, potassium chloride, potassium phosphate, and sodium phosphate were purchased from Sigma as reagent grade or higher purity and used without any additional preparation. PBS was prepared as 1× (˜[10 mM]). AlexaFluor 405 NHS ester was purchased from Invitrogen. CypHer 5E NHS ester was purchased from GE Life Sciences. Chromatography grade DMSO (Sigma) was dried through a column of molecular sieves (4 Å, Sigma) under dry nitrogen gas. Water was distilled and deionized to 18.6 MΩ using a Barnstead Diamond RO Reverse Osmosis machine coupled to a Barnstead Nano Diamond ultrapurification machine. Anti-cubilin antibodies (ab65773) were purchased from Abcam (Cambridge, Mass.). The tetanus toxoid was a gift from the Serum Institute of India (2400 L_{f}/mL; lot number 1170).

Temperature-dependent MD simulations for the tetanus neurotoxin light chain (LC) have been performed using GROMACS (v. 4.0.4, Journal of Computational Chemistry 2005, 26, 1701) with the GROMOS96 (53a6, Biomolecular Simulation: The GROMOS96 Manual and User Guide, Vdf Hochschulverlag AG an der ETH Zürich, Zürich, 1996) united-atom force field (the same force field for which B12 parameters have been generated and used in the two previous studies). The procedure for the temperature-dependent studies involves 50 nanosecond (ns) simulations of each protein within center-of-mass (COM)-defined periodic boundary condition (PBC) constraints, with the system temperatures of 300 and 400 K used for this series of simulations. All simulations are performed as NPT (constant particle number, constant pressure, constant temperature) ensembles with 1.0 femtosecond (fs) time-steps. The (X,Y,Z) dimensions of the water boxes are defined as four times the length of the longest atom-atom separation in each protein (for the LC, this results in a solvent box containing 12,344 water molecules) to provide for an adequate initial unfolding volume without appreciable protein-protein interactions within the PBC-constrained system volume. Simulation temperatures for each of the steps in the MD simulations are kept constant using Berendsen thermostats with 0.1 picosecond (ps) coupling time constants for solvent (using the flexible simple point charge (SPC) water model and neutralizing sodium (Na⁺) counterions) and solute, separately. Simulation pressures are kept constant using isotropic Berendsen barostats of 1.0 bar with a time coupling constant of 0.5 ps. Electrostatic interactions are evaluated by the particle mesh Ewald (PME) method order 6 with a grid spacing of 0.1 nm in the X,Y,Z directions. The real-space and neighbor-search cutoffs (which define the distances over which two atoms not joined by force field stretch or bend terms have their interaction energies calculated in the total electrostatic contribution to the system energy) are set to 1.2 nm, with this non-bonded pair lists updated every 10 steps.

Gel permeation purification was performed on an Agilent 1200 HPLC with manual injection and automated fraction collector fitted with a Zorbax GF-450 analytical column (9.4×250 mm) and a Zorbax GF-250 analytical column (4.6×250 mm) in series. 0.11 M NaCl was used as the mobile phase at a flow rate of 1 mL/min. MALDI-TOF mass spectrometry was performed on Applied Biosystems Voyager-DE with a laser intensity of 3922 Hz. The MALDI matrix was 10 mg sinapinic acid dissolved in a 70:30 water:acetonitrile mixture with 0.1% TFA.

All in vitro cell experiments were preformed in an air-filtered, UV-irradiated Labconco Purifier I laminar flow hood. RPMI 1640 was purchased from the American Type Culture Collection (ATCC). Fetal bovine serum and cell stripper were purchased from Mediatech of Manassas, Va. Penicillin-Streptomycin solution with 10,000 units of penicillin and 10 mg per mL streptomycin in 0.9% NaCl was purchased from Sigma. The BeWo choriocarcinoma human cell line (ATCC code CCL-98) was obtained from the ATCC. Cell cultures were grown in a mammalian cell incubator with constantly maintained humid environment and 5% CO₂. Cells were grown in BD Falcon 250 mL culture bottles with vented lids with 30 mL of media. Subcultures were passed by the following protocol: Used media was discarded, and the flask was rinsed with 2 mL of fresh media. The adherent cultures were removed from the flask by incubating with 3 mL of Cellstripper for 20 minutes. The suspension was then centrifuged down and the liquid was decanted. The cells were then suspended in 5 mL of media and passed with a split ratio of 1/10. All cell lines were grown in RPMI 1640 media with 10% FBS and 10% penicillin-streptomycin. The prepared media was filtered with a 0.22 μm sterile filter (VWR). Conjugate concentrations were calculated using Bradford assay (BioRad, Mass.) using BSA as standard. TT light chain graphics were generated using VMD and POVRay (Persistence of Vision Raytracer, v.3.6.1, www.povray.org) on a 1.5 GHz processor (Powerbook G4, Apple, Inc.).

Confocal microscopy experiments were conducted with a Zeiss LSM 700 Pascal confocal microscope equipped with Ar and HeNe lasers. Images were analyzed using the Zeiss Zen image analysis software (2008). Excitation was conducted at 405 nm for Alexaflouro 405 conjugates and 600 nm for CypHer 5E conjugates. HPLC was used to confirm stability over the time frame of the confocal experiments.

Synthesis of B₁₂-TT Conjugates 1 and 2. B₁₂ (40 mg, 0.03 mmol) and CDT (5 mg, 0.03 mmol) were dissolved in 3 mL of DMSO. The reaction was heated to 70° C., under N₂, and stirred for one hour. This solution was added in three aliquots over a span of one hour to the TT (2400 Lf) in 2 mL of 0.05 M carbonate buffer, pH 9.6. The final amount of activated solution of B₁₂ that was added varied depending on the mole ratio desired. The amount of B₁₂ that was added to the TT was either 20 mg (0.015 mmol) or 1 mg (0.0007 mmol). The reaction was stirred overnight at 4° C. The reaction was first purified using dialysis (MWCO 50,000) against 0.11 M NaCl, for 6×1L over two days.

Bradford assay. The Quick Start Bradford Assay was purchased from Fischer and was performed according to the manufacturer's instructions (BioRad). Briefly, 5 μl of each sample was placed into a 96 well plate in triplicate. 250 μL of quick start Bradford dye reagent was added to each well. The plate was allowed to incubate for 10 minutes at room temperature. The absorbance was read at 670 nm. The absorbance was compared to a calibration curve based on known concentrations of BSA (2 mg/mL-0.15 mg/mL) run concurrently.

ELISA assay. 100 μl of 1 μg/mL rabbit antibody solution (ab53829; polyclonal to TT) in 0.05 M carbonate buffer (pH 9.6) was incubated in 96 well plate overnight at 4° C. The solution was removed and washed three times with phosphate buffered saline, pH 7.4, 0.05% tween (PBST). After each of the following incubations, the plate was washed as described. 200 μL of PBST with 2.5% dried milk (PBSTM) was added to the wells, and was incubated for one hour at 37° C. 100 μl of the compounds/TT standard dilutions were incubated for two hours at 37° C. Each concentration was done in triplicate. 100 μl (0.8 μg/mL) of mouse antibody solution (ab26247; monoclonal to TT) in PBS™ were incubated for two hours at 37° C. 100 μl (1 μg/mL) of rabbit antibodies (ab6728; polyclonal to mouse IgG) conjugated to horseradish peroxidase in PBS™ was incubated for 1 h at 37° C. After the final wash, 100 μl of 2,2′-azino-bis(3-ethylbenzothiazoline-6-sulfonic acid) liquid substrate was added to the wells and was allowed to incubate for 15 min at room temperature. The absorbance at 405 nm was then recorded. The control concentrations of TT were plotted and fit least square regression. The calibration curve was only considered accurate when the line had an R² value greater than 0.85. The best fit line was used to calculate the concentration of the samples.

Cubilin antibody Tagging with Alexa Fluor 405 (Cub Ab₄₀₅). Cubilin antibody (ab65773) (10 μg) was added to 20 μL of carbonate buffer, pH 9.6. To this solution, 10 μg of Alexa Fluor 405 was added in 50 μL of DMSO. The reaction rotated at room temperature for one hour, protected from light. The reaction was then dialyzed (MWCO 50,000) against PBS pH 7.4 (3×1 L) over 24 h.

TT and B₁₂-TT CypHer 5E Tagging (TT_{C5E}, 1_{C5E}, 2_{C5E}). B₁₂-TT conjugates or TT was dissolved in carbonate buffer, pH 9.6 (200 μg/mL). To the solution, 10 μg of CypHer 5E dissolved in DMSO was added. The reaction was rotated in the dark for 2 h at room temperature. The solution was then dialyzed overnight into PBS, pH 7.4 at 4° C. The samples were kept at 4° C., protected from light until incubation.

IF Alexa Fluor 405 Tagging (IF₄₀₅). IF (10 mg) was added to 1 mL of carbonate buffer, pH 9.6. The solution was vortexed and centrifuged to remove undissolved particulate. To this solution, 50 μL of Alexa Fluor 405 (50 μg) solution in DMSO was added. The reaction was rotated at room temperature for one hour protected from light. The reaction was then dialysized (MWCO 25,000) against PBS buffer pH 7.4 (3×1 L) over 24 h.

Fluorescent immunostaining of cubilin. The BeWo cells were plated (200,000 cells/dish) on 356×100 mm vented dishes. The cells were incubated at 37° C. overnight in RPMI 1640 media. To each plate was added 500 μl of CubAb₄₀₅ dissolved in PBS. The plate was then incubated for one hour. The drug was then removed, and the cells were washed with 1×PBS (3×1 mL) at a pH of 7.4. The cells were then washed with PBS at 3.5 (3×1 mL) to prevent any noncovalent membrane interaction with the proteins. The cells were passed back into PBS buffer, pH 7.4 and viewed under the confocal microscope.

Confocal microscopy confirmation of B₁₂-TT uptake. The BeWo cells were plated (200,000 cells/dish) on 356×100 mm vented dishes. The cells were incubated at 37° C. overnight in RPMI 1640 media. To each plate was added 1 mL of 1_{C5E} or 2_{C5E} in a solution of IF [1 mg per mL] in PBS. The plate then incubated for 1 h. The drug was then removed, and the cells were washed with 1×PBS (3×1 mL) at a pH of 7.4. The cells were then washed with PBS at 3.5 (3×1 mL) to prevent any noncovalent membrane interaction with the proteins. The cells were placed back into PBS buffer, pH 7.4 and viewed under the confocal microscope.

Confocal microscopy confirmation of 1_{C5E}, 2_{C5E} and IF₄₀₅ colocalization. The BeWo cells were plated (200,000 cells/dish) on 356×100 mm vented dishes. The cells were incubated at 37° C. overnight in RPMI 1640 media. To each plate was added 1 mL of 1_{C5E} or 2_{C5E} with IF₄₀₅ in PBS. The plate then incubated for one hour. The drug was then removed, and the cells were washed with PBS (3×1 mL) at a pH of 7.4. The cells were then washed with PBS at pH 3.5 (3×1 mL) to prevent any reduce membrane interaction with the proteins. The cells were placed back into PBS buffer, pH 7.4 and viewed under the confocal microscope.

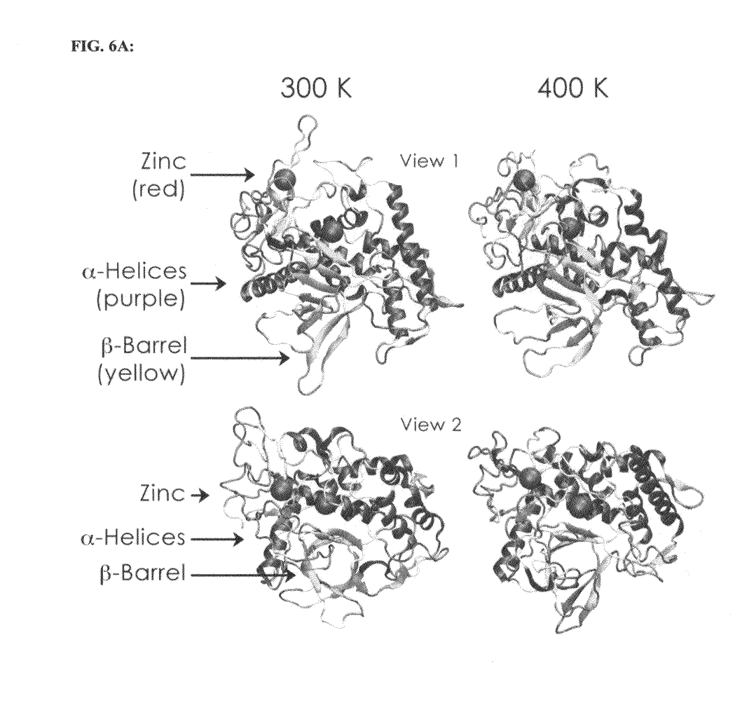

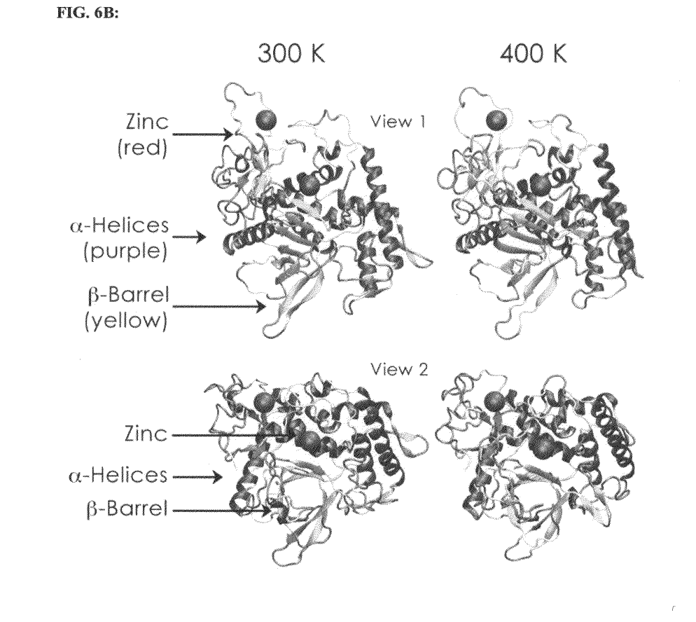

Results. FIGS. 6A and 6B show the results of Applicants' molecular dynamics simulations. FIG. 6A shows the geometries for both LC forms at their initial time steps, including the positions of the two zinc ions (red) identified in the crystallographic study. The key regions of the tetanus LC from a molecular recognition perspective are the b-barrel (in yellow) localized to one side of the LC and the α-helical region (purple) within which over half of the β-barrel is embedded. Ignoring much of the complexity of side chain positions and flexible loops (green and white in the images), the retention of the α/β arrangement and the overall stability of this larger motif over the course of the simulations is of considerable significance.

FIG. 6B shows the same LC structures (and rotated views) after 40 ns. The similarity of these two images, despite the considerable difference in simulation temperatures (the 400 K temperature is, after all, 47 K higher than the formalin treatment used in vaccine manufacture and 27 K higher than the point at which water boils), is remarkable. The geometry of the 300 K secondary α/β-structure is largely unaffected (and it must be noted that these are simply time step snapshots, meaning the structure is undergoing thermal motion) and the 400 K simulation, despite some apparent deformation of the β-barrel away from cylindrical symmetry, still retains a great deal of the 300 K geometry, including the relative positions of the zinc ions and much of the α-helical region. Statistical analyses of the geometries indicate that nearly all of the flexibility at both temperatures is localized to unstructured loops and not the α/β-structure. This study then reveals that the secondary structure is likely very stable and not subject to unfolding-refolding as part of the vaccine manufacturing process and lends further support to the hypothesis that the stable geometry of the α/β-secondary structure may itself be key in immune response.

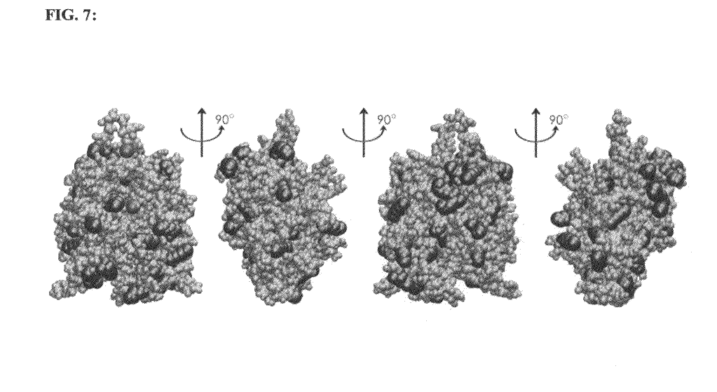

Tetanus Light Chain Solvent-Accessible Lysine Surface Distribution: In light of the remarkable apparent stability of the tetanus LC from Applicants' 300 K and 400 K MD studies, it was plausible to begin to map the possible locations of B₁₂ upon amide linkage formation at accessible lysine side chains. As the LC α/β-secondary structure is found to be remarkably stable, the surface-accessible lysine residues from the crystallographic determination of the LC geometry now become the only significant group of lysine residues to consider in the B₁₂ bioconjugate design work. This solvent-accessible map of lysine residues is shown against the entire LC in FIG. 7. As can be seen, the LC contains a plethora of lysine side chains (in blue) that are solvent-accessible against a solvent surface rendering of the entire LC protein (in yellow; zinc ions are shown in red).

### Synthesis of Vitamin B₁₂-Tetanus Toxoid (B₁₂-TT) Conjugate

The molecular dynamics simulations suggested that the TT may function as a conformational epitope, with minimal unfolding occurring even at elevated temperatures. This allowed us to predict a coupling route for B₁₂ to TT and allowed us to rationalize that adding several B₁₂ molecules, as opposed to a 1:1 ratio, may not greatly interfere with TT activity (especially also given the changes that occur upon detoxification).

B₁₂ was activated using CDT at 60° C. in dry DMSO. The activated B₁₂ was added in aliquots over one hour to the TT in 50 mM carbonate buffer at pH 9.6. The TT is rich in lysine residues as exemplified by FIG. 4, which shows the light chain (50 kDa) of the TT with the lysine residues in cyan as van der Waals, making CDT an ideal conjugation route. Two different amounts [1 mg (0.007 mmol) or 20 mg (0.0148 mmol)] of activated B₁₂ were reacted with 2400 L_{f} of TT to give conjugates 2 and 1.

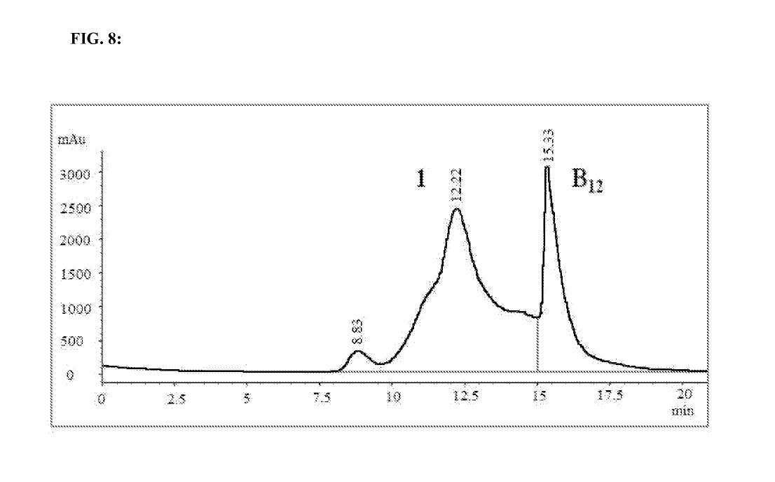

Purification. Purification of the conjugated system was achieved using gel permeation chromatography (see FIG. 8). The different synthetic ratios of B₁₂ gave similar chromatographic behavior, with the TT clearly dominating the separation behavior.

The weight of the TT itself was initially established and was noted at ˜158 kDa by mass spectrometry. The additional 8 kDa on the predicted 150 kDa (based on amino acid sequence) most likely comes from the incorporation of formalin and lysine during the inactivation process. This established a baseline molecular weight and was used to compare to the new peaks from the HPLC separation. The mass spectrometry also showed free separate light and heavy chains in the TT sample.

The molecular weight of conjugate 1 was approximately 170 kDa. This is 12 kDa higher than the experimentally observed weight of the TT. The mass increase from the free TT to the conjugated TT is equivalent to nine B₁₂ molecules. By using a reaction with a smaller synthetic ratio of B₁₂ to TT, a molecular weight of 163 kDa was observed by mass spectrometry. For 2, compared to the free TT again, the shift in molecular weight was only 5 kDa. This is consistent with addition of four molecules of B₁₂.

To further characterize the conjugates a tryptic digest was performed on the conjugates. The digest was purified by reverse phase HPLC and the fractions were lyopholized, and subsequently redissolved for MALDI analysis. It is important to note that each fraction contained multiple peaks. The MALDI results were compared to the expected digest. Peptide fragments that showed conjugation of B₁₂ are listed below (Table 1).

TABLE 1
Molecular weights, position and sequence
of suspected fragments of conjugation
(Weight of peptide only).

| Molecule | | |
|---|---|---|
| Weight | Position | Amino Acid |
| (Da) | (Chain) | Sequence |
| 882 | 405-411 (L) | DLKSEYKGQNMR |
| 882 | 86-92 (L) | FLQTMVK |
| 940 | 1007-1014 | FNAYLANK |
| | (HC) | |
| 1003 | 721-727 | RSYQMYR |
| | (HN) | |
| 1143 | 99-109 (L) | NNVAGEALLDK |
| 1291 | 1211-1222 | VGYNAPGIPLYK |
| | (HC) | |
| 1382 | 334-345 (L) | DSNGQYIVNEDK |
| 1384 | 97-109 (L) | IKNNVAGEALLDK |
| 1777.9 | 417-433 (L) | VNTNAFRNVDGSGLVSK |
| 2069.9 | 602-619 | DIIDDFTNESSQKTTIDK |
| | (HN) | |
| 3007 | 560-584 | ITMTNSVDDALINSTKI |
| | (HN) | YSYFPSVISK |

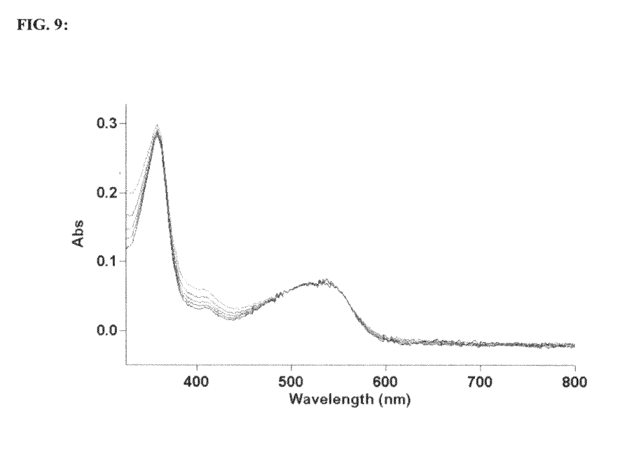

In vitro activity of B₁₂. The first in vitro tests conducted were conducted to confirm that conjugation did not disrupt biological recognition of B₁₂. The first was binding of intrinsic factor (IF, a glycosylated protein produced in the gastric cells of the stomach) to the B₁₂-TT conjugate. Intrinsic factor is the protein that facilitates intestinal uptake and so is critical if delivery of the TT is to be successful to the blood stream. Binding of IF to B₁₂ has been shown to increase the extinction coefficient in electronic absorption spectra. An electronic absorption spectrum of fraction 1 from the HPLC separation (retention time of 9.3 minutes) is shown in FIG. 9. Sequential aliquots of IF were added to the B₁₂-TT, and the absorbance of the compound increased (see FIG. 9).

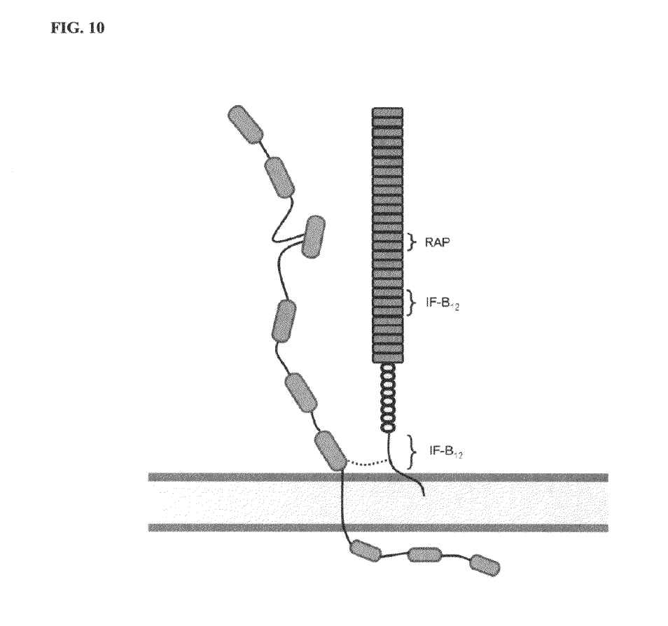

Once the binding to intrinsic factor was confirmed, we sought to prove that human cells containing the cubilin receptor could internalize the B₁₂-TT. Cubilin is a ˜460 kDa protein composed of eight epidermal growth factor domains and 27 CUB domain proteins (see FIG. 10). CUB domains are typically ˜110-115 amino acid residues in size and are derived from Clr/Cls, Uegf, and bone morphogenic protein-1 complements. Binding of IF-B₁₂ to cubilin occurs primarily at CUB domains 5-8, and appears to be calcium dependent. This receptor is expressed in the gastrointestinal tract and is the entry route for B₁₂ to the blood stream. If the B₁₂-TT is to be delivered it must pass through this receptor, and then it is taken up by the epithelial cells of the ileum through a megalin-mediated process.

The very size limitation of the B₁₂ uptake pathway had not previously been explored. In arriving at the instant invention, the successful in vitro uptake of B₁₂-tetanus toxoid conjugates (MW>160 kDa) through the cubilin receptor, was achieved. This represents a significant breakthrough, as TT is the primary component of the tetanus vaccine, is a therapeutic, immunologically active protein of large size (>150 kDa), and has previously required subcutaneous administration, despite significantly need for a more readily-accessible therapeutically active dosage form.

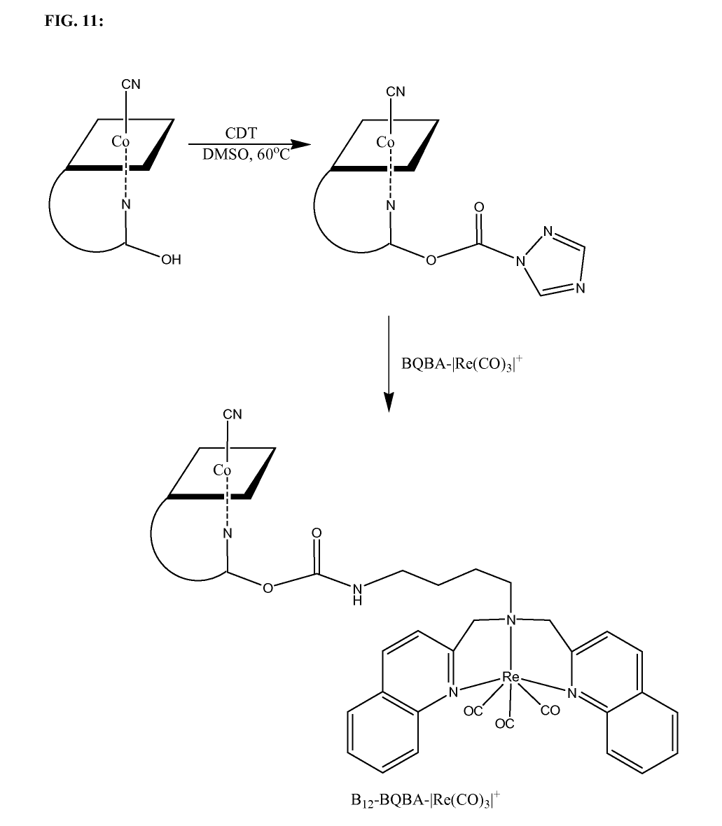

To investigate whether B₁₂-TT could be transported by cubilin we designed and synthesized a fluorescent probe, B₁₂-BQBA-[Re(CO)₃]⁺ (2), for cubilin (see FIG. 11) based on the fluorescent properties of rhenium(I) complexes. We used this probe to investigate whether the human placental cell line, BeWo, expressed the cubilin receptor and so would be suitable for subsequent B₁₂-TT studies.

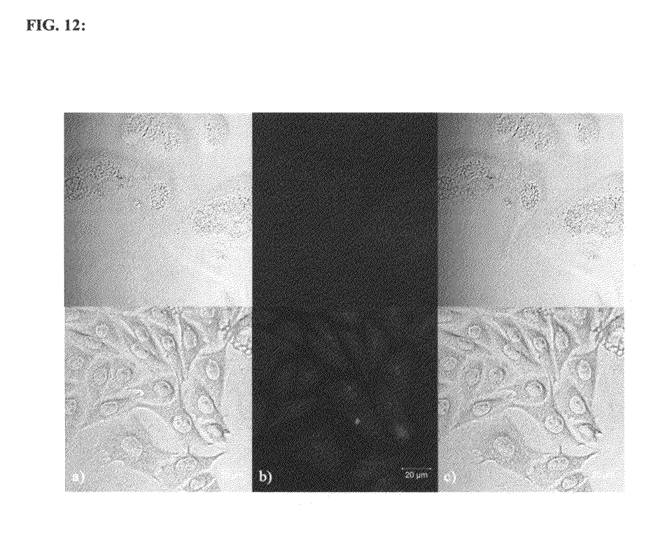

Uptake experiments via fluorescent confocal microscopy of B₁₂-Rhenium probe (2). To the BeWo cell line, a total volume of ˜1.5 ml of 10 μM 2 (bound to IF as followed by electronic absorption spectroscopy and designated here as 2-IF) was added. The cells were then incubated at 37° C. over two separate periods of ˜45 minutes or 6 hours to allow probe influx. The solution of 2-IF was then removed via pipette and the cells washed with RPMI 1640 media and 1×PBS buffer (pH ˜7.4) three times. Intracellular fluorescence was observed after 45 minutes of drug exposure (see FIG. 12). This indicates rapid entry of the drug into the cells and is consistent with the presence of the cubilin receptor in the BeWo cells.

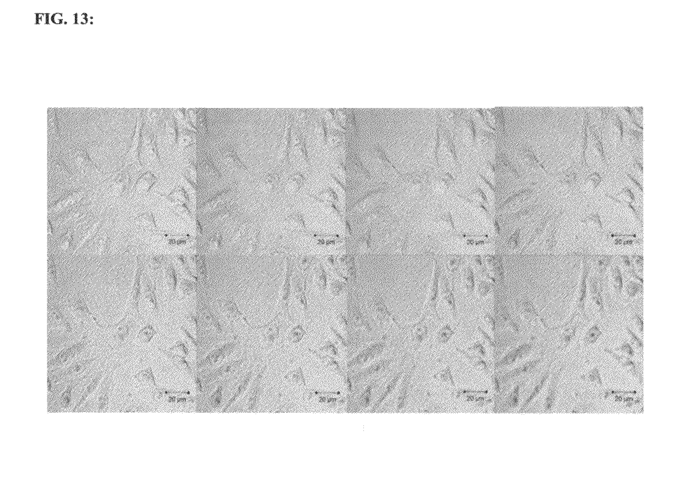

To further prove that internalization indeed occurred, optical slicing at ˜1 μm per slice was conducted as a gallery view (see FIG. 13). Fluorescence in the cellular milieu was only observed in the middle sections, confirming the internalization of 2-IF.

Competitive binding with B₁₂ against 2-IF was investigated by adding excess of the vitamin. Excess B₁₂ at concentrations of ˜[100 μM] and ˜[10 mM] were added first to the cells and incubated at RT for ˜5 min. Then, 2-IF was added to the cells. The final concentration of 2-IF was ˜[10 μM]. The cells were then incubated for ˜45 minutes, after which, the media containing 2-IF and B₁₂ was discarded. The cells were then washed with media 1×PBS (pH ˜7.4) in triplicate to remove residual 2-IF. Based on observations, addition of a hundred-fold excess [100 μM] B₁₂ did little to block binding of 2-IF complex to the cubilin receptor. At higher concentrations of B₁₂ ([10 mM]) however, cellular access and accumulation of 2-IF was completely inhibited. In addition, there was no observed fluorescence even after 6 hrs of incubation under these conditions, indicating that the excess B₁₂ was clearly inhibiting uptake of 2-IF. To further support evidence that IF is critical for the B₁₂-conjugate transport and that entry is gained through the cubilin receptor, the BeWo cells were exposed to 2 unbound to IF (i.e. 2 only) and with prior addition of [10 mM] excess B₁₂ to determine uptake. No fluorescence was observed at 45 minutes and even after 6 hrs for cells with prior exposure to excess B₁₂. By saturating the cells with a thousand-fold excess of B₁₂ however, we noted that transport of 2 was shut down, presumably since now no free IF is available and the fact that the B₁₂-transport proteins have a greater affinity for the unmodified B₁₂.

siRNA gene knockdown of cubilin receptor. To fully establish the route of uptake as cubilin based, small interfering RNA (siRNA) specific for cubilin mRNA was transfected into the BeWo cells to knockdown expression of the cubilin receptor. siRNA mediated inhibition of cubilin with corresponding loss of uptake of 1-IF would provide conclusive evidence that internalization of 2-IF in BeWo cells proceeds via the cubilin receptor uptake pathway and establishes 2 as a specific bioprobe for this receptor. A fluoroscein conjugate of random sequence siRNA, known not to inhibit any mRNA, was employed as a transfection uptake marker. After transfection of the cubilin siRNA, the cells were incubated at 37° C. Uptake experiments were conducted after allowing the cells to grow over 24 hours. The growth medium was aspirated and [10 μM] 2-IF was added to the transfected cells and control cells (no siRNA of any type added) and incubated over 45 minutes. After incubation, the drug-containing medium was discarded and the cells were then washed with 1×PBS in triplicate to remove residual 2-IF. At both time points, confocal microscopy experiments showed illumination for both control and transfected cells. Fluorescence intensity values differed markedly however. The control plate displayed a mean intensity of ˜4.0, whereas the transfected cells had an average recorded intensity value of ˜0.4. This significant decrease (˜10-fold) in fluorescence intensity, by as much as six fold, in the transfected cells is indicative of a significant reduction of drug uptake, and correlates with knock down of the cubilin gene. We also conducted uptake experiments after 48 hours of transfection. Optical slices (˜1 μm) of the transfected BeWo cells revealed internalization with varying fluorescence intensities ranging from 0.81 to as much as 3.7, consistent with the cubilin receptor switching back to expression upon multiple passages (the BeWo line has a doubling time of 20 hrs).

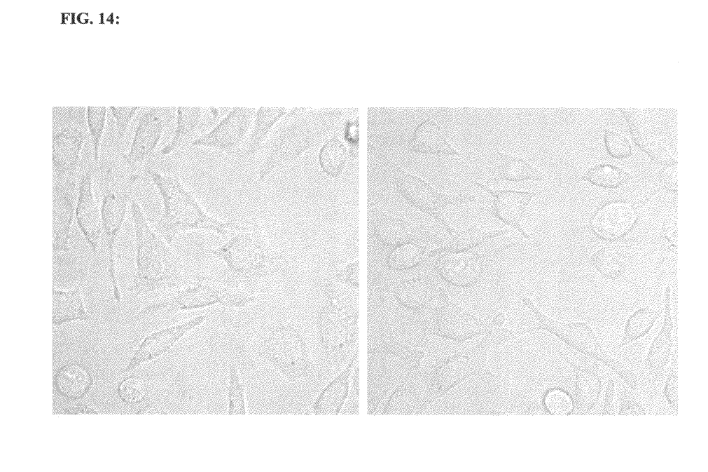

Having established the BeWo line as an excellent model for cubilin receptor based transport we conducted uptake studies with B₁₂-TT. Since the B₁₂-TT system is not inherently fluorescent we had to build a fluorescent B₁₂-TT system. This was achieved by conjugation of the B₁₂-TT system with CypHer 5E dye. This dye turns purple and fluoresces when the environmental pH is less than 6. If uptake occurs and is receptor mediated by endocytosis, the pH will drop to 5.5 and we would see purple fluorescence. The new B₁₂-TT-CypHer system was incubated with IF, then was incubated with the BeWo cells for 45 minutes. The cells were examined with confocal microscopy to look for uptake and internalization. The compound was found in the cells (see FIG. 14). Using optical slices, the fluorescence was confirmed internalized in the cell and is not due to random surface adhesion.

At this point we had successfully produced B₁₂ bound TT and established a viable purification method. The new system was recognized by Intrinsic factor and was still transported by cubilin. Critically, this implies the B₁₂ uptake pathway is still fully functional even with bound TT.

Biological activity of the B₁₂-TT conjugate. The next round of work focused on activity of the TT itself: To confirm the biological activity of the conjugate, an ELISA was designed at Syracuse University. Rabbit polyclonal anti-TT antibodies were conjugated to 96 well plates. After incubating TT dilution standards (240-0.002 Lf/mL), different concentrations of mouse monoclonal anti-TT (1-0.5 μg/mL) were incubated to determine the appropriate dilution needed for a valid assay. Secondary antibodies conjugated to horseradish peroxidase were then incubated in the wells. The assay used a concentration range 1 L_{f}/ml to 0.005 L_{f}/mL that was found to generate a linear calibration curve. The calibration line however was only linear up to ˜10 L_{f}/mL concentrations of TT. In order to make sure that the conjugate's and the control's ELISA results are comparable, the concentration of protein in each sample was determined by Bradford assay.

Combining the Bradford results with the ELISA results, the samples are presented as ratio of L_{f} to μg. This number was compared to a Bradford assay done on the TT starting material, which gave an L_{f} to μg ratio of 0.32. The ratio of L_{f}/μg in the conjugates is lower than the TT starting material.

Both conjugates were then exposed to IF and binding was followed by electronic absorption spectroscopy as described previously. In both cases, IF binding was noted as indicated by an increase in absorption.

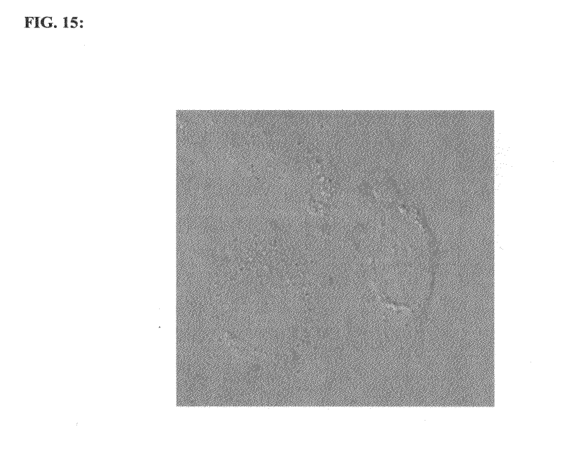

To establish a baseline for the presence of cubilin in the BeWo cell line and complement our earlier work in this area, cubilin immunostaining was conducted. Antibodies to cubilin were tagged with Alexa Fluoro 405 dye (CubAb₄₀₅). The antibodies were then dialyzed for 24 hours to remove excess dye, followed by HPLC. The BeWo cells were incubated with CubAb₄₀₅ for 45 min at 37° C., and then examined with confocal microscopy (see FIG. 15). The cells show binding to the surface and some internalization of CubAb₄₀₅. The CubAb₄₀₅ was also incubated against the Chinese hamster ovary cell line (CHO). CHO cells do not express the cubilin receptor and therefore are a suitable negative control. The CubAb₄₀₅ did not show binding or uptake to the CHO cells (data not shown). The results of the immunostaining are consistent with those previously reported on the presence of cubilin in BeWo cells.

In order to rule out the possibility of TT mediated uptake in the BeWo cells, the TT was conjugated to the AlexaFluoro 405 tag (TT₄₀₅). TT₄₀₅ was incubated with the BeWo cell line with IF present. The BeWo cells did not take up the TT₄₀₅, to support the B₁₂ mediated uptake hypothesis. The TT₄₀₅ did show some slight membrane interaction but critically no internalization. Given the transmembrane nature of the heavy chain of the TT, this interaction is consistent with the reported TT mode of action.

With confirmation that the cell line contains cubilin and that TT does not facilitate uptake, 1 and 2 were conjugated with CypHer 5E dye to make fluorescent conjugates (1_{C5E}, 2_{C5E}). CypHer 5E was chosen for these conjugates because under neutral conditions, the tag is fluorescently silent. When exposed to an acidic pH such as 5.5, it fluoresces. Observing fluorescence would suggest that the uptake is proceeding through receptor mediated endocytosis. 1_{C5E} was initially incubated with IF for 30 minutes, then incubated with BeWo cells for 1 hour.

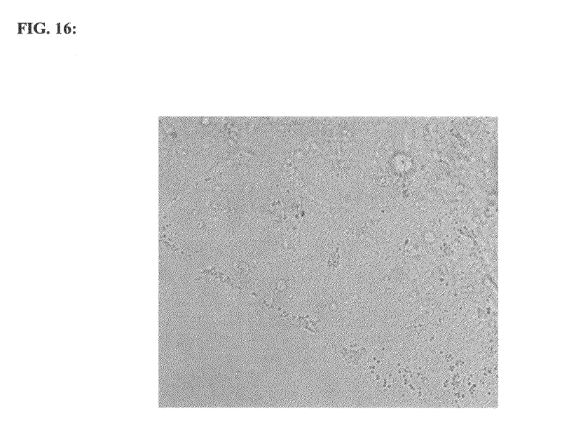

The cells were then washed with PBS at pH of 7.4 (×3) and at a pH of 3.0 (×1) to wash away free conjugate and reduce nonspecific membrane interactions. PBS at pH 7.4 was then added to the cells, and they were examined with confocal microscopy to look for uptake and internalization (see FIG. 16). 1_{C5E} clearly demonstrates internalization in the cells, which was further shown by 1 nm optical slices.

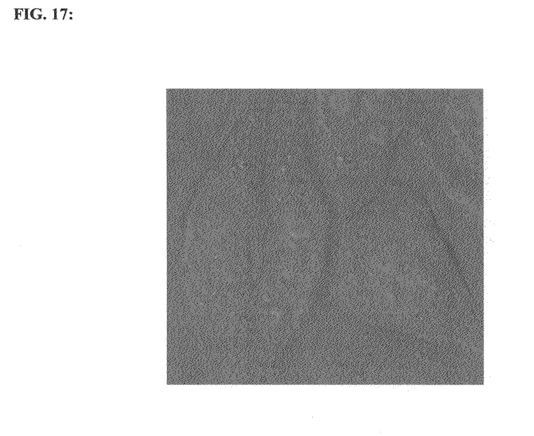

To confirm an IF mediated uptake of the B₁₂ tetanus conjugate, a colocalization experiment was conducted with fluorescently-tagged IF (IF₄₀₅). IF was reacted with the Alexa Fluor 405 for one hour and then dialyzed for 24 h to give the tagged IF₄₀₅ system. The IF₄₀₅ system was incubated in the presence of B₁₂, 1_{C5E} and 2_{C5E} conjugates. The Alexa Fluor 405 fluorophore was chosen because its excitation and emission profile does not overlap with the CypHer 5E dye. At least a 10-fold excess of IF was used throughout. The IF₄₀₅ incubated with B₁₂ shows uptake in the BeWo cells (see FIG. 17).

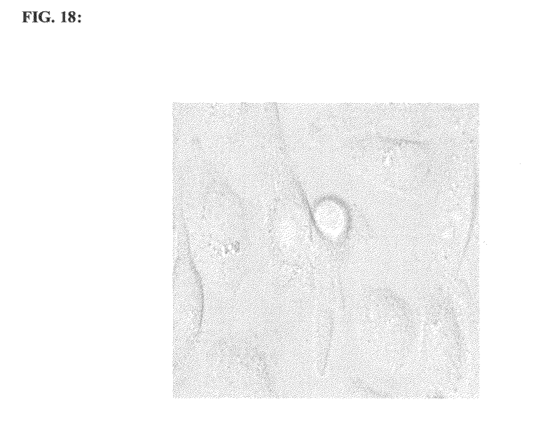

When the IF₄₀₅ was incubated with 1_{C5E} there was uptake and colocalization of the red and blue signals, confirming both IF and TT were present together. The blue signal commonly surrounds the red from the conjugate (see FIG. 18). This is consistent with multiple B₁₂ molecules being conjugated around the TT, which then are bound by IF₄₀₅.

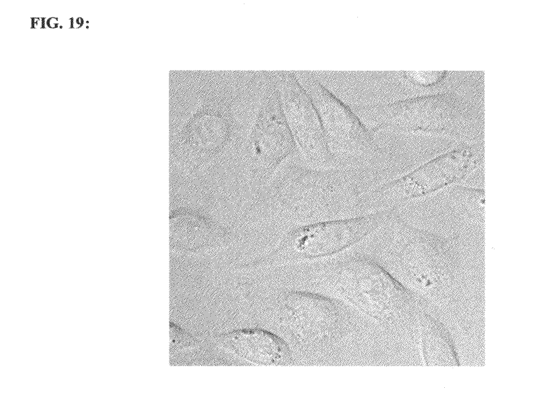

The 2_{C5E} conjugate also showed uptake and colocalization, but the enveloping effect of IF₄₀₅ noted for 1_{C5E} was greatly reduced and greater uptake was observed (see FIG. 19). This is consistent with the lower number of B₁₂s conjugated to the TT making up 2_{C5E}, thereby resulting in less IF₄₀₅ binding.

Building off of previous work which demonstrated that the BeWo human placental line takes up a fluorescently tagged B₁₂ molecule in a cubilin dependent mechanism, Applicants decided to test the hypothesis that uptake of a large, fluorecently-tagged protein could be followed in vitro in the BeWo cell line. This would simulate the intestinal uptake of the conjugate in vivo and suggest if the B₁₂ uptake pathway could accommodate large proteins, such as toxoids, polysaccharide polymers or antibodies for therapeutic administration.

Conjugation of B₁₂ to such a large target proved to be a complex proposition. The experimental observations back up the MD simulations with multiple conjugations of B₁₂ to the TT. The trypsin digestion of the conjugates show more possible sites of conjugation than the parental weight of the conjugates suggest. This suggests that the resulting conjugates are not a single species, but rather a mixture of conjugates containing B₁₂ located at different residues. This polymorphism is most likely due to the inherent polymorphism of the TT starting material itself, and due to the simple conjugation chemistry with the abundance of conjugation sites. This polymorphism causes issues when trying to purify and characterize these conjugates. However, the polymorphism can be used to help explain the ELISA results.

While the ELISA shows a marked decrease (100 fold) activity of the conjugate when compared to the TT starting material, there are important considerations to keep in mind. The primary antibody used was a monoclonal antibody. This means that if a B₁₂ molecule blocked that epitope, the conjugate would appear silent, regardless of the amount of other exposed antigenic epitopes. In an in vivo setting, the loss of antigencity may be far less, due to the compensation of the other antigenic epitopes. Despite the decrease in antigenicity, the conjugates still show activity in the ELISA, which means that antibody recognition can still occur despite conjugation.

To support the hypothesis that the B₁₂ uptake pathway could carry large proteins, the conjugate, IF, and anti-cubilin antibodies were followed via confocal microscopy. The Cub showed binding to the target cells (BeWo) while it did not show binding to the control cells (CHO). This further backs up the choice of the BeWo cell line as an appropriate cubilin model. The IE₄₀₅ conjugate shows uptake in the BeWo cell line. This also supports the choice of the model for uptake studies. The 1_{C5E} and 2_{C5E} conjugates are both seen internalized in the cells as well. The fluorescence of these conjugates suggests a receptor mediated endocytosis uptake, due to the pH sensitive CypHer 5E dye. The TT_{C5E} conjugate which shows no uptake confirms that the TT alone is not able to induce receptor mediated endocytosis. This suggests that the B₁₂ conjugation is required for the endocytosis of the conjugates. This is important because tetanus toxin is able to induce endocytosis in neuron cells. This result is important because if the TT itself could cross the enterocyte, there would be no need for further modification.

The colocalization of the IF₄₀₅ with the conjugate systems further support the cubilin receptor mediated endocytosis. While there are some areas that do not show the conjugate, everywhere a conjugate is present, the IF is also present. This is presumably due to either untagged conjugate, or free B₁₂ left from the medium.

The work herein shows that it is possible for the uptake pathway to transport a 170 kDa conjugate system. This work indicates that certain large proteins, such as some vaccines and antibodies, including TT, can be delivered as orally active conjugates.

While several aspects of the present invention have been described and depicted herein, alternative aspects may be effected by those skilled in the art to accomplish the same objectives. Accordingly, it is intended by the appended claims to cover all such alternative aspects as fall within the true spirit and scope of the invention.

What is claimed is:

1. An orally active vitamin B₁₂-tetanus toxoid (TT) conjugate.

2. The orally active vitamin B₁₂-TT conjugate of claim 1, wherein TT is covalently attached to the primary (5′) hydroxyl group of the ribose moiety of vitamin B₁₂.

3. The orally active vitamin B₁₂-TT conjugate of claim 1 wherein vitamin B₁₂ is directly or indirectly attached to TT via surface-accessible lysine residues on TT.

4. The orally active vitamin B₁₂-TT conjugate of claim 1 wherein any attached Vitamin B₁₂ molecule resides on the exterior of one or more of the tetanus subunits such that the overall tetanus geometry is maintained.

5. The orally active vitamin B₁₂-TT conjugate of claim 1, wherein the conjugate exhibits at least a portion of the immunogenicity of TT.

6. The orally active vitamin B₁₂-TT conjugate of claim 1, wherein the conjugate comprises a carbamate-linkage.

7. The orally active vitamin B₁₂-TT conjugate of claim 1, wherein the conjugate further comprises a spacer group between the vitamin B₁₂ and TT.

8. The orally active vitamin B₁₂-TT conjugate of claim 7, wherein said spacer group comprises a polyethylene glycol monomer spacer unit.

9. The orally active vitamin B₁₂-TT conjugate of claim 7, wherein said spacer group comprises two or more polyethylene glycol monomer spacer units.

10. A pharmaceutical composition comprising the orally active vitamin B₁₂-TT conjugate of claim 1 and a pharmaceutically acceptable carrier or diluent.

11. The pharmaceutical composition of claim 10, further comprising Intrinsic Factor.

12. The pharmaceutical composition of claim 11, wherein the Intrinsic Factor is human Intrinsic Factor.

13. The pharmaceutical composition of claim 10 wherein said orally active vitamin B₁₂-TT conjugate is encapsulated in protective liposomes.

14. A pharmaceutical composition, comprising:
an oral delivery form of TT comprising vitamin B₁₂ covalently coupled to TT, wherein the covalent coupling is between a dicarboxylic acid derivative of the primary (5′) hydroxyl group of the ribose moiety of vitamin B₁₂ and surface-accessible lysine residues of TT, and a pharmaceutically acceptable carrier suitable for oral delivery,
wherein the pharmaceutical composition exhibits immunogenic activity when delivered orally to a mammal.

15. A method of preventing tetanus disease, comprising orally administering to a patient in need thereof an immunologically effective amount of the orally active B₁₂-TT conjugate of claim 1.

16. The method of claim 15, wherein the pharmaceutical composition is in an oral delivery form selected from the group consisting of a liquid, a capsule, a tablet, an emulsion, a colloidal dispersion, an elixir, a gel and a paste.

17. The method of claim 15, wherein the TT is covalently attached to the primary (5′) hydroxyl group of the ribose moiety of vitamin B₁₂.

18. The method of claim 15, wherein vitamin B₁₂ is attached to TT via surface-accessible lysine residues of TT.

19. A kit comprising the pharmaceutical composition of claim 10 and an instruction sheet for oral administration.

20. A kit comprising the pharmaceutical composition of claim 14 and an instruction sheet for oral administration.
